(19) United States
(12) Patent Application Publication
(10) Pub. No.: US 2025/0195560 A1
(43) Pub. Date: Jun. 19, 2025

(54) PHARMACEUTICAL COMPOSITION INCLUDING HYALURONIC ACID OR SALT THEREOF AND APPLICATION THEREOF

(71) Applicant: Beijing Zhongyan Chuangke Medical Technology Co., Ltd., Beijing (CN)

(72) Inventor: WEIHENG REN, Beijing (CN)

(21) Appl. No.: 18/410,849

(22) Filed: Jan. 11, 2024

(30) Foreign Application Priority Data
Dec. 13, 2023 (CN) 202311708752.9

(51) Int. Cl. A61K 31/728; A61P 17/00

(52) U.S. Cl. CPC A61K 31/728; A61P 17/00

(57) ABSTRACT

The present disclosure provides a pharmaceutical composition including a hyaluronic acid or a salt thereof and an application thereof. The present disclosure includes a composition including a low molecular weight microbial fermentation of the hyaluronic acid or the salt thereof, an excipient, and a glycosylglycerol or a derivative thereof. An addition amount of the composition for the low molecular weight microbial fermentation of the hyaluronic acid or the salt thereof is 0.001%-15%, an addition amount of the glycosylglycerol or the derivative thereof is 0.001%-15%. The glycosylglycerol used in the pharmaceutical composition of the present disclosure mainly comes from desert selaginella or salt tolerant blue-green algae, the composition of a low molecular weight microbial fermentation of a hyaluronic acid or a salt thereof has been used for many years in fields such as medicine and daily chemicals, it is safe and effective and can effectively treat Th2 type skin inflammation.

### CROSS-REFERENCE TO RELATED APPLICATIONS

This application claims priority to Chinese Patent Application No. 202311708752.9, filed on Dec. 13, 2023, which is hereby incorporated by reference in its entirety.

### TECHNICAL FIELD

The present disclosure relates to the field of medical technologies, and in particular, to a pharmaceutical composition including a hyaluronic acid or a salt thereof and an application thereof.

### BACKGROUND

In immunology, CD4+ T cells can be divided into Th1 and Th2 cell subsets based on a pattern of cytokine secretion. Th1 cells mediate cellular immunity, cytotoxic T lymphocyte (CTL), macrophage activation, and delayed hypersensitivity reactions. Th2 mediated humoral immunity, activation of B cells and eosinophils, and generation of IgE, and its characteristic lies in an elevated level of IgE antibodies and an increase in eosinophils. Th2 type cells mainly secrete IL-4, IL-5, IL-6, IL-10, and IL3, known as Th2 cytokines. Under a normal circumstance, Th1/Th2 cells are in a relatively balanced state, maintaining positive cellular and immune functions in the body. But when the body experiences functional abnormalities, it often shows a balance leaning towards one side. In some immune diseases, different diseases exhibit different Th1/Th2 balance shifts. When Th2 cells dominate, it can trigger Th2 related diseases. Th2 type inflammatory skin diseases include atopic dermatitis, psoriasis, chronic idiopathic urticaria (eczema), bullous pemphigoid, nodular prurigo, scabies, chronic idiopathic pruritus, eosinophilic dermatitis, insect bite dermatitis, etc. Research has shown that TSLP is an upstream target of the inflammatory cascade in Th2 diseases, and thymic stromal lymphopoietin (TSLP) is an epithelial derived cytokine that binds to the TSLP receptor TSLPR to exert biological functions. After binding to TSLP-TSLPR, it activates dendritic cells to shift Th2 towards Th1, giving Th2 cells an advantage and triggering Th2 type diseases. Due to TSLP being an upstream target of the inflammatory cascade, there is an opportunity to address related diseases from the source before other downstream disease-related cytokines such as IL-4, IL-5, IL-13, IL-17, and IgE have an impact.

At present, existing topical drugs for Th2 skin diseases mainly rely on glucocorticoids, while other effective treatments include Benvimod for psoriasis and anti-human interleukin-8 monoclonal antibodies. Due to a fact that most Th2 skin diseases belong to long-term chronic diseases, a long-term medication is required. After long-term use, glucocorticoids have significant side effects and can cause Cushing's syndrome, digestive symptoms, metabolic disorders, and exacerbate inflammatory reactions, causing harm to human health, especially in children. Due to an extensive effect of glucocorticoids on growth, metabolism, and immunity, there is a lack of a clear target for Th2 type diseases, resulting in poor efficacy. However, other topical drugs generally have limitations on side effects such as dosage, application area, and cumulative use time, and their costs are extremely high.

Based on this, a pharmaceutical composition including a hyaluronic acid or a salt thereof and an application thereof is provided, which provides a solution to address the aforementioned issues.

### SUMMARY

A purpose of the present disclosure is to provide a pharmaceutical composition including a hyaluronic acid or a salt thereof and an application thereof to solve an existing problem, the existing topical drug targeting Th2 type skin diseases, glucocorticoids are mainly used, while other effective drugs include Benvimod for treating psoriasis and anti-human interleukin-8 monoclonal antibodies. Due to the fact that most Th2 skin diseases belong to long-term chronic diseases, and a long-term medication is required. After long-term use, glucocorticoids have significant side effects and can cause Cushing's syndrome, digestive symptoms, metabolic disorders, and exacerbate inflammatory reactions, causing harm to human health, especially in children. Due to an extensive effect of glucocorticoids on growth, metabolism, and immunity, there is a lack of a clear target for Th2 type diseases, resulting in poor efficacy. However, other topical drugs generally have limitations on side effects such as dosage, application area, and cumulative use time, and their costs are extremely high.

To solve the above-mentioned technical problems, the present disclosure is implemented through the following technical solutions.

The present disclosure provides a pharmaceutical composition including the hyaluronic acid the salt thereof, which includes a composition of a low molecular weight microbial fermentation of the hyaluronic acid or the salt thereof, an excipient, and a glycosylglycerol or a derivative thereof. An addition amount of the composition of the low molecular weight microbial fermentation of the hyaluronic acid or the salt thereof is 0.001%-15%, an addition amount of the glycosylglycerol or the derivative thereof is 0.001%-15%.

In an embodiment of the present disclosure, a molecular weight of the composition of the low molecular weight microbial fermentation of the hyaluronic acid or the salt thereof is 10 Da-50 KDa.

Another object of the present disclosure is to provide an application of the above-mentioned pharmaceutical composition including the hyaluronic acid or the salt thereof in a preparation of a drug for treating thymic stromal lymphocyte mediated skin diseases.

In an embodiment of the present disclosure, an application of the pharmaceutical composition including a hyaluronic acid or a salt thereof in a preparation of a drug for treating atopic dermatitis.

In an embodiment of the present disclosure, an application of the pharmaceutical composition including a hyaluronic acid or a salt thereof in a preparation of a drug for treating psoriasis.

In an embodiment of the present disclosure, an application of the pharmaceutical composition including a hyaluronic acid or a salt thereof in a preparation of a drug for treating pruritus.

In an embodiment of the present disclosure, an application of the pharmaceutical composition including a hyaluronic acid or a salt thereof in a preparation of a drug for treating eczema.

In an embodiment of the present disclosure, an application of the pharmaceutical composition including a hyaluronic acid or a salt thereof in a preparation of a drug for treating skin allergies.

The present disclosure has the following beneficial effects:

The glycosylglycerol used in the pharmaceutical composition of the present disclosure is a glycoside compound formed by connecting one molecule of glycerol and one molecule of glucose through glycosidic bonds. It mainly comes from a plant such as desert selaginella or salt tolerant blue-green algae and is a plant extract; the composition of low molecular weight microbial fermentation of hyaluronic acid or a salt thereof has been used for many years in fields such as medicine and daily chemicals. It has been proved by long-term practice that it is safe and effective, has no side effects on human body, can relieve skin irritation, can be applied on the skin surface for a long time and in a large area, can effectively treat Th2 type skin inflammation, atopic dermatitis, psoriasis, pruritus and eczema, and can be used in a variety of dosage forms, can be formulated as a liquid dosage form, a gel dosage form, a cream dosage form or a lotion dosage form, with a wide application range.

### BRIEF DESCRIPTION OF DRAWINGS

In order to provide a clearer explanation of the technical solution of the embodiments of the present disclosure, a brief introduction will be given below to the drawings required for a description of the embodiments. It is obvious that the drawings in the following description are only some embodiments of the present disclosure. For those skilled in the art, other drawings can be obtained based on these drawings without creative work.

FIG. 1 is a bar chart of a relative expression level of tslpr gene of the present disclosure.

FIG. 2 is a diagram of a typical total motion distance relief of the present disclosure.

### DESCRIPTION OF EMBODIMENTS

The following will provide a clear and complete description of the technical solution in the embodiments of the present disclosure, in combination with the drawings. Obviously, the described embodiments are only a part of the embodiments of the present disclosure, not all of them. Based on the embodiments in the present disclosure, all other embodiments obtained by ordinary technicians in the art without creative work fall within the protection scope of the present disclosure.

In this embodiment, a pharmaceutical composition including a hyaluronic acid, or a salt thereof includes a composition of a low molecular weight microbial fermentation of the hyaluronic acid or the salt thereof, an excipient, and a glycosylglycerol or a derivative thereof.

Moreover, an addition amount of the composition of the low molecular weight microbial fermentation of the hyaluronic acid or the salt thereof is 0.001%-15%, an addition amount of the glycosylglycerol or the derivative thereof is 0.001%-15%.

Where, glycosylglycerol is a glycoside compound formed by connecting one molecule of glycerol and one molecule of glucose through glycosidic bonds.

Here, a molecular weight of the composition of the low molecular weight microbial fermentation of the hyaluronic acid or the salt thereof is 10 Da-50 KDa.

The glycosylglycerol used in this embodiment is extracted from desert selaginella or salt tolerant blue-green algae and synthesized without a use of chemical enzymatic catalysis.

Applying the above-mentioned pharmaceutical composition in a preparation of a drug for treating thymic stromal lymphocyte mediated skin diseases, atopic dermatitis, psoriasis, pruritus, eczema, and skin allergies.

After experimental verification, the pharmaceutical composition of the present disclosure can effectively treat immune skin diseases mediated by TSLP (thymic stromal lymphocytes), or skin itching, redness, and inflammation related to such diseases, such as atopic dermatitis, psoriasis, pruritus, eczema, skin allergies, skin adverse reactions after tumor radiotherapy and chemotherapy, etc.

It can also be used in a variety of skin external dosage forms, which can be prepared into powder, gel, emulsion, paste and other ointment, film, rehydratable freeze-dried paste or sponge, sprayable solution, locally coated adhesive and liquid storage tank system, coated intermediates (such as thin films and bandages), film matrix or flexible polymer matrix.

### Experiment 1

### Experimental Method

1. Randomly selecting zebrafish in a 6-well plate, with 30 fish per well.
2. Administering water soluble SLS to establish a zebrafish skin inflammation model.
3. Administering a pharmaceutical composition of the present disclosure in water and setting up a normal control group and a model control group, with a capacity of 3 mL per well; three biological replicates.
4. Incubating under a dark condition at 4.28° C. for 18 hours.
5. Extracting total RNA from zebrafish in each experimental group, synthesizing cDNA, and detecting gene expression of β-actin and target genes by q-PCR.
6. Using β-actin as an internal reference for gene expression, calculating a relative RNA expression level of the target gene.

$${RNA}_{relative expression level} = {2}^{ΔΔ C ( t )}$$

$$ΔΔ C ( t ) = Δ {C ( t )}_{Model control group} - Δ {C ( t )}_{Sample group}$$

$$Δ C ( t ) = {C ( t )}_{Target gene} - {C ( t )}_{β - actin} .$$

### Experimental Principle:

Sodium dodecyl sulfonate (SLS) may trigger a stimulating response in the body, which leads to symptoms such as edema, erythema, pruritus, and pain. A stimulating phenotype of the skin is mainly manifested as skin inflammation, with early inflammation mainly manifested as capillary dilation, hyperpermeability, and edema. Stimulants enter the body of zebrafish, which induces an inflammatory response. TSLP (Thymic Stromal Lymphopoietin) is a pleiotropic cytokine. A biological role of TSLP mainly depends on its binding to receptors. An affinity between TSLPR (TSLP receptor) and TSLP is very low, a high affinity receptor complexes is formed only with IL7R α (α chain of IL7 receptor), and a ternary complex is formed. This ternary complex can cause phosphorylation of JAK (non receptor tyrosine kinase) and STATs (signal transduction and transcriptional activation proteins), thereby initiating inflammatory response related signaling pathways. It can be seen that TSLP-TSLPR and downstream signaling molecules play an important role in an occurrence and development of inflammation. Therefore, a relative expression level of tslpr gene can be detected to evaluate whether the pharmaceutical composition of the present disclosure has an inhibitory effect on TSLPR (TSLP receptor).

### Determination Basis:

Statistical analysis showed p<0.05, determining there is a significant difference.

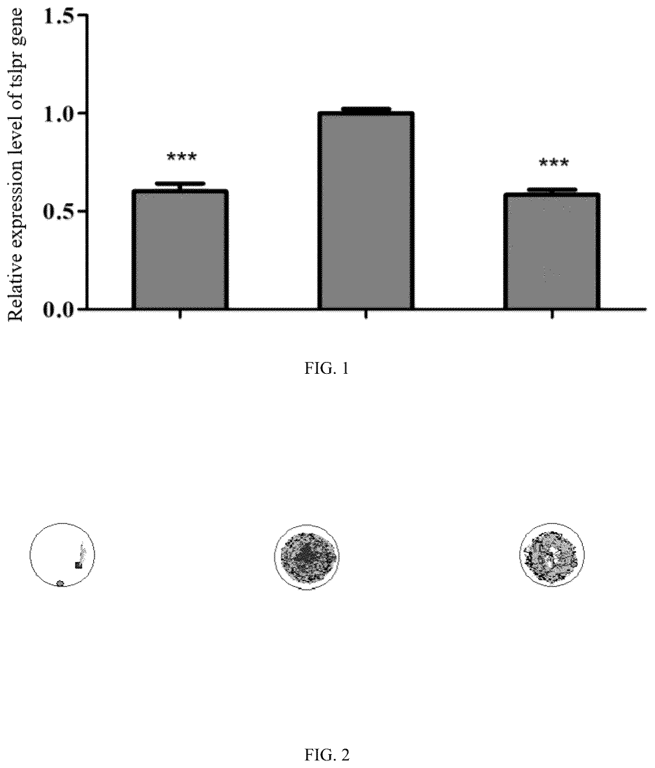

Please refer to FIG. 1, which shows a normal control group, model control group, and a pharmaceutical composition of the present disclosure from left to right.

Observation shows that a relative expression level of the gene in the pharmaceutical composition of the present disclosure is significantly reduced compared to the model control group, which reveals that the pharmaceutical composition of the present disclosure has an inhibitory effect on TSLPR (TSLP receptor).

TABLE 1

| | | Relative | | |
|---|---|---|---|---|
| | Detection | expression | | |
| | concentration | level of | | Detection |
| Testing items | (%) | tslpr gene | P value | result |
| Inhibitory effect | 0.5 | 0.584 | <0.001 | significant |

### Experimental Conclusion:

Under conditions of this experiment, the pharmaceutical composition of the present disclosure has a significant effect on TSLP (thymic stromal lymphopoietin).

### Experiment 2:

### Experimental Method:

1. Randomly selecting zebrafish in a 6-well plate, with 15 fish per well.
2. Administering the pharmaceutical composition of the present disclosure in water and setting up a normal control group and a model control group, with a capacity of 3 mL per well.
3. Incubating under a dark condition at 3.28° C. for 24 hours.
4. Ten zebrafish were randomly selected from each experimental group and transferred to a 96 well plate. Water soluble acetic acid was administered to establish a zebrafish pain model, a behavior analyzer was immediately used to measure a movement trajectory of zebrafish, a total movement distance (D) of zebrafish is analyzed and collected, a soothing effect of the pharmaceutical composition according to a formula is calculated, and whether there is a soothing effect is determined.

$$soothing effect ( \% ) = \frac{D ( model control group ) - D ( sample group )}{D ( model control grou ) - D ( normal control grou )} \times 100 \%$$

### Experimental Principle:

Although acetic acid is a weak acid, it has strong penetrating and corrosive properties. When the skin contacts with acetic acid, it can cause a tingling and burning sensation. Zebrafish with underdeveloped swimming ability is chosen and acetic acid was used to stimulate zebrafish, which causes larger and longer inflammatory pain, resulting in more movement trajectories and more severe pain, a behavior analyzer was used to detect a total movement distance of zebrafish and whether the pharmaceutical composition of the present disclosure has the soothing effect is evaluated.

### Determination Basis

Statistical analysis showed that p<0.05, determining there is a significant difference.

TABLE 2

| | Detection | | | |
|---|---|---|---|---|
| | concentration | | | Detection |
| Testing items | (%) | Effect (%) | P value | result |
| Soothing effect | 0.5 | 26 | <0.01 | significant |

The efficacy experiment of the pharmaceutical composition of the present disclosure is shown in FIG. 2, they are the normal control group, model control group, and the pharmaceutical composition of the present disclosure form left to right. The red line represents a distance of rapid movement, the green line represents a distance of moderate movement, and the black line represents a distance of slow movement.

Observation shows that a total movement distance of the pharmaceutical composition of the present disclosure is significantly reduced compared to the model control group, revealing that the pharmaceutical composition of the present disclosure has the soothing effect.

### Experimental Conclusion:

Under conditions of this experiment, the total movement distance of the pharmaceutical composition of the present disclosure was significantly reduced compared to the model control group, which reveals that the pharmaceutical composition of the present disclosure has the soothing effect.

In summary, the pharmaceutical composition of the present disclosure has no toxic side effects on the human body, can quickly relieve skin irritation, can be applied to the surface of the skin for long-term and large area, and can effectively treat Th2 type skin inflammation, thymic stromal lymphocyte mediated skin diseases, atopic dermatitis, psoriasis, pruritus, eczema, and skin allergies. The target of action is clear, and the inhibition rate of inflammatory factors is high.

What is claimed is:

1. A pharmaceutical composition including a hyaluronic acid or a salt thereof, comprising a composition of a low molecular weight microbial fermentation of the hyaluronic acid or the salt thereof, an excipient, and a glycosylglycerol or a derivative thereof;
an addition amount of the composition of the low molecular weight microbial fermentation of the hyaluronic acid or the salt thereof is 0.001%-15%, an addition amount of the glycosylglycerol or the derivative thereof is 0.001%-15%.

2. The pharmaceutical composition including a hyaluronic acid or a salt thereof according to claim 1, wherein a molecular weight of the composition of the low molecular weight microbial fermentation of the hyaluronic acid or the salt thereof is 10 Da-50 KDa.

3. An application of the pharmaceutical composition including a hyaluronic acid or a salt thereof according to claim 1 in a preparation of a drug for treating thymic stromal lymphocyte mediated skin disease.

4. An application of the pharmaceutical composition including a hyaluronic acid or a salt thereof according to claim 2 in a preparation of a drug for treating thymic stromal lymphocyte mediated skin disease.

5. The application of the pharmaceutical composition including a hyaluronic acid or a salt thereof according to claim 1 in a preparation of a drug for treating atopic dermatitis.

6. The application of the pharmaceutical composition including a hyaluronic acid or a salt thereof according to claim 2 in a preparation of a drug for treating atopic dermatitis.

7. The application of the pharmaceutical composition including a hyaluronic acid or a salt thereof according to claim 1 in a preparation of a drug for treating psoriasis.

8. The application of the pharmaceutical composition including a hyaluronic acid or a salt thereof according to claim 2 in a preparation of a drug for treating psoriasis.

9. The application of the pharmaceutical composition including a hyaluronic acid or a salt thereof according to claim 1 in a preparation of a drug for treating pruritus.

10. The application of the pharmaceutical composition including a hyaluronic acid or a salt thereof according to claim 2 in a preparation of a drug for treating pruritus.

11. The application of the pharmaceutical composition including a hyaluronic acid or a salt thereof according to claim 1 in a preparation of a drug for treating eczema.

12. The application of the pharmaceutical composition including a hyaluronic acid or a salt thereof according to claim 2 in a preparation of a drug for treating eczema.

13. The application of the pharmaceutical composition including a hyaluronic acid or a salt thereof according to claim 1 in a preparation of a drug for treating skin allergies.

14. The application of the pharmaceutical composition including a hyaluronic acid or a salt thereof according to claim 2 in a preparation of a drug for treating skin allergies.
